(19) United States
(12) Patent Application Publication
(10) Pub. No.: US 2025/0114512 A1
(43) Pub. Date: Apr. 10, 2025

(54) CLAMP MECHANISM FOR OPHTHALMIC SURGICAL CONSOLES AND CASSETTES

(71) Applicant: Alcon Inc., Fribourg (CH)

(72) Inventor: Vincent A. Baxter, Temecula, CA (US)

(21) Appl. No.: 18/882,981

(22) Filed: Sep. 12, 2024

Related U.S. Application Data
(60) Provisional application No. 63/587,793, filed on Oct. 4, 2023.

(51) Int. Cl. A61M 1/00

(52) U.S. Cl. CPC A61M 1/72; A61M 2205/121

(57) ABSTRACT

Embodiments disclosed herein provide a clamp assembly for engaging a surgical cassette with an ophthalmic surgical console. The clamp assembly includes a motion plate and a plurality of clamp mechanisms coupled to the motion plate. A plurality of bases which remain in a fixed position are also coupled to each of the plurality of clamp mechanisms and to a face plate portion of a fluidics subsystem of the ophthalmic surgical console. A motion plate motor is provided which serves to move the motion plate in a vertical direction relative to the face plate. As the motion plate is vertically displaced, the clamp mechanisms coupled thereto are opened and expanded into the surgical cassette, thereby pressing it against the face plate and ensuring any connections and seals between the face plate and the surgical cassette are established and maintained.

### BACKGROUND

Ophthalmic surgical procedures are often classified as anterior segment surgical procedures, posterior segment procedures, or combined anterior segment and posterior segment procedures (i.e., “combined procedures”). The anterior segment refers to the front-most region of the eye, and includes the cornea, iris, and lens. Thus, anterior segment surgical procedures typically include surgeries performed on the iris and/or lens, such as cataract surgery. The posterior segment refers to the back-most region of the eye that includes the anterior hyaloid membrane and the optical structures behind it, such as the vitreous humor, the retina, the choroid, and the optic nerve. Posterior segment surgical procedures typically include retinal and vitreoretinal surgeries. In certain cases, a patient may have pathologies of the eye requiring both anterior and posterior procedures; in such cases, a combined procedure may be performed. During anterior and/or posterior segment surgery, tissue fragments and other materials may be aspirated or suctioned out of the eye through, e.g., a hollow needle or cannula. Also during the procedure, an irrigating or infusion fluid may be pumped into the eye to maintain an intraocular pressure (IOP) and prevent collapse of the eye.

During ophthalmic surgery, a surgical cassette having one or more peristaltic and/or venturi pumps, as well as one or more valve assemblies, may be used to facilitate the aspiration and irrigation functionalities described above. Generally, the surgical cassette is connected to a surgical console by first placing the cassette into a fluidics module of the surgical console and engaging the cassette with a plurality of hooks, snap-fit joints, or other locking mechanisms. However, conventional surgical cassette locking mechanisms have a number of significant shortcomings when applied to new fluidic cassette technologies requiring better sensor stability and higher clamping forces, including failing to maintain a secure connection between the surgical cassette and the surgical console. A loose surgical cassette can lead to many problems during a surgical procedure, such as failed or inaccurate fluid sensor readings, failure to maintain irrigation and aspiration pump head engagement with the surgical cassette, and/or failure to maintain engagement with the various cassette valves and seals, among other issues.

### BRIEF SUMMARY

The present disclosure relates generally to ophthalmic surgical consoles and cassettes, clamp assemblies used to maintain engagement between the surgical consoles and cassettes, and methods of use thereof.

In certain embodiments, a clamp assembly is provided for engaging a surgical cassette with an ophthalmic surgical console. The clamp assembly includes a motion plate and a plurality of clamp mechanisms having a distal end and a proximal end, the distal end of each of the plurality of clamp mechanisms being coupled to the motion plate. A plurality of bases are in turn coupled to the proximal end of each of the plurality of clamp mechanisms. Additionally, a plurality of guides are also coupled to the plurality of clamp mechanisms. In certain embodiments, each of the plurality of clamp mechanisms comprises a pair of hooks that are disposed at each of their respective proximal ends.

In certain embodiments, the clamp assembly includes a guide (also referred to as a guide bracket) with a mount hole for a fixed pin (also referred to as a base pin), a first guide channel, and a second guide channel. Two opposed hook arms (also referred to as bodies) are connected near the center with a pivot pin (also referred to as a guide pin) so that they can rotate about the pivot pin. The pivot pin also goes through first guide channel of the guide bracket to constrain the pivot pin motion to front to back and back to front. A return spring (also referred to as a torsion spring) is configured to apply a return force to the pivot pin acting from the guide. A control pin (also referred to as a clamp pin) goes through slots (also referred to as angled tracks) defined at the distal end of the hook arms and through the secondary guide channel of the guide, so that the control pin is constrained to only move in the distal or proximal directions. The control pin is mounted to a first linkage arm (also referred to as a linkage) near the back of the assembly and is configured to move the control pin. Additionally, a spacer or spacer bushing may be used to fit over the control pin through slots of the hook arms and between the guide and the first linkage arm to ensure space and prevent binding.

Movement of the control pin from a proximal position near the front of the surgical console to a distal position near the back of the surgical console pushes against the slots of the hook arms and rotates the hook arms, which “expands” or “contracts” the distance between the hooks and thus engages or disengages with the cassette. The first linkage arm is mounted at the other end to a linkage arm assembly defined by a first lever, a second lever, and a lever pin, with a linkage pin to allow relative rotation. The linkage arm assembly is mounted to the fixed pin near the front of the assembly. The linkage arm assembly can extend or compress between its two connecting points at the fixed pin and the linkage pin and provides expansion force by use of a clamp spring. Movement upwards of the lever pin acts to rotate the linkages and displace the control pin distally, which in turn expands the hooks of the hook arms. Alternatively, an actuation pin (also referred to as a plate pin) may be added at another location on the first linkage arm or linkage arm assembly and used to actuate the clamp arm assembly mechanism.

In certain embodiments, mounting of the clamp arm assembly mechanism is accomplished through mounting holes for screws in a base (also referred to as a base bracket). The base is attached to the fixed pin, which in turn secures the guide. The base can be arranged to be opposite of the guide from the two hook arms to provide additional space for other module and cassette features. In this arrangement, the fixed pin protrudes through a recess in the hook arms to connect the guide to the base and supports one connecting point of the linkage arm assembly.

In certain embodiments, the clamp arm assembly mechanism can be actuated by use of a motion plate linked to the actuation pin. In certain embodiments, multiple clamp arm assembly mechanisms can be mounted and actuated.

In certain embodiments, a clamp mechanism may include a guide (having a mount hole for a base pin, a first guide channel, and a second guide channel). The clamp mechanism may further include two opposed bodies comprising hooks. The two opposed bodies may be connected with a guide pin such that the two opposed bodies are configured to rotate about the guide pin. The guide pin may be disposed through the first guide channel to constrain motion of the guide pin. The two opposed bodies may include angled tracks at respective distal ends of each of the opposed bodies. The clamp mechanism may further include a return spring configured to apply a return force to the guide pin acting from the guide and a clamp pin disposed through the angled tracks at the distal ends of each of the opposed bodies and through the secondary guide channel of the guide to constrain motion of the clamp pin. In some embodiments, the clamp mechanism may further include a linkage with the clamp pin mounted to the linkage. The linkage may be configured to move the clamp pin. In some embodiments, movement of the clamp pin from a proximal position to a distal position pushes against the angled tracks of each opposed body and rotates each opposed body to change a distance between the hooks. Changing the distance between the hooks acts to engage or disengage a surgical cassette. In some embodiments, the clamp mechanism may further include a spacer placed over the clamp pin and through the respective angled tracks of each opposed body and between the guide and the linkage. In some embodiments, the clamp mechanism may further include a linkage arm assembly with a first lever, a second lever, and a lever pin. The linkage may be mounted at an opposing end to the linkage arm assembly with a linkage pin disposed therein to allow relative rotation. In some embodiments, the linkage arm assembly is mounted to the base pin and can extend or compress between the base pin and the linkage pin. The linkage arm assembly may be configured to provide an expansion force from a clamp spring. Upward movement of the lever pin acts to rotate the linkages and displace the clamp pin distally to expand the hooks of each opposing body until the clamp pin reaches an end of the angled tracks. Further movement of the lever pin may move the hooks distally with a surgical cassette until the surgical cassette contacts a faceplate supporting the base pin. Further movement of the lever pin may compress the linkage arm assembly between the base pin and the linkage pin and change a length of the clamp spring.

In some embodiments, the clamp mechanism may further include a plate pin on the linkage or the linkage arm assembly that is configured to actuate the clamp mechanism. In some embodiments, a clamp assembly may include one or more of these clamp mechanisms with a motion plate (having a motion slot) in connection to each plate pin of each clamp mechanism. The clamp assembly may further include a motor and a drive wheel assembly comprising a motion pin (engaged in the motion slot). In some embodiments, the motor may be configured to rotate the motion pin by rotating the drive wheel assembly. In some embodiments, rotation of the motion pin moves the motion plate substantially linearly such that the motion plate actuates the one or more clamp mechanisms. The motion slot may include a grooved bushing that is configured to engage the motion pin of the drive wheel assembly.

The following description and the related drawings set forth in detail certain illustrative features of one or more embodiments.

### BRIEF DESCRIPTION OF THE DRAWINGS

The appended figures depict certain aspects of one or more disclosed embodiments and are therefore not to be considered limiting of the scope of this disclosure.

FIG. 1A illustrates a front view of an exemplary ophthalmic surgical system used to perform ophthalmic procedures on an eye, according to certain embodiments.

FIG. 1B illustrates a plan view of exemplary subsystems of a surgical console of the ophthalmic surgical system of FIG. 1A, according to certain embodiments.

FIG. 1C illustrates cassette slots for engagement with the hooks of the clamp assembly, according to certain embodiments.

FIG. 2A illustrates a frontal perspective view of a clamp assembly for engaging a surgical cassette with a surgical console, e.g., the surgical console of FIGS. 1A and 1B, according to certain embodiments.

FIG. 2B illustrates a rear perspective view of the clamp assembly of FIG. 2A, according to certain embodiments.

FIG. 3 illustrates a frontal perspective view of a clamp sub-assembly of the clamp assembly of FIG. 2A, the clamp sub-assembly comprising an upper clamp mechanism and a lower clamp mechanism, according to certain embodiments.

FIG. 4 illustrates an exploded view of the clamp sub-assembly of FIG. 3, according to certain embodiments.

FIG. 5 illustrates a frontal perspective view of the clamp assembly of FIGS. 2A and 2B when installed on a fluidics subsystem of an ophthalmic surgical console, according to certain embodiments.

FIG. 6 illustrates a frontal perspective view of the clamp assembly when installed on the fluidics subsystem of the ophthalmic surgical console of FIG. 5 with a bezel removed from the fluidics subsystem and exposing a face plate disposed beneath, according to certain embodiments.

FIG. 7 illustrates an exploded view of the fluidics subsystem of the ophthalmic surgical console with the clamp assembly of FIG. 5, according to certain embodiments.

FIG. 8A illustrates a perspective view of the clamp assembly of FIGS. 2A and 2B coupled to a motion plate motor via a rotatable drive wheel with motion pin, according to certain embodiments.

FIG. 8B illustrates a frontal view of the clamp assembly, motion plate motor, and the rotatable drive wheel with motion pin of FIG. 8A, according to certain embodiments.

FIG. 8C illustrates a side perspective view of the clamp assembly, motion plate motor, and the rotatable drive wheel with motion pin of FIG. 8A, according to certain embodiments.

FIG. 9A illustrates a frontal view of the clamp assembly of FIGS. 8A-8C after a motion plate has been lifted upward in the vertical direction by the rotatable drive wheel with motion pin, the movement of the motion plate opening each of a plurality of clamp mechanisms within the clamp assembly, according to certain embodiments.

FIG. 9B, illustrates a side perspective view of the actuated clamp assembly of FIG. 9A, according to certain embodiments.

FIG. 10A illustrates a frontal view of the clamp assembly of FIGS. 9A and 9B after the motion plate has been lifted further upward in the vertical direction by the rotatable drive wheel with motion pin, the movement of the motion plate further opening each of a plurality of clamp mechanisms within the clamp assembly, according to certain embodiments.

FIG. 10B, illustrates a side perspective view of the actuated clamp assembly of FIG. 10A, according to certain embodiments.

FIG. 11A illustrates a frontal view of the clamp assembly of FIGS. 10A and 10B after the motion plate has been lifted further upward in the vertical direction by the rotatable drive wheel with motion pin, the movement of the motion plate moving each of a plurality of clamp mechanisms within the clamp assembly in a distal direction, according to certain embodiments.

FIG. 11B, illustrates a side perspective view of the actuated clamp assembly of FIG. 11A, according to certain embodiments.

FIG. 12A illustrates a frontal view of the clamp assembly of FIGS. 11A and 11B after the motion plate has been lifted further upward in the vertical direction by the rotatable drive wheel with motion pin, the movement of the motion plate further moving each of a plurality of clamp mechanisms within the clamp assembly in the distal direction while expanding a corresponding plurality of springs within each of the plurality of clamp mechanisms, according to certain embodiments.

FIG. 12B, illustrates a side perspective view of the actuated clamp assembly of FIG. 12A, according to certain embodiments.

FIG. 13A illustrates a frontal view of the clamp assembly of FIGS. 12A and 12B after the motion plate has been lifted further upward in the vertical direction by the rotatable drive wheel with motion pin, the movement of the motion plate further expanding the corresponding plurality of springs within each of the plurality of clamp mechanisms, according to certain embodiments.

FIG. 13B illustrates a side perspective view of the actuated clamp assembly of FIG. 13A, according to certain embodiments.

FIG. 14A illustrates a magnified perspective view of one of a plurality of clamp mechanisms within the clamp assembly of FIGS. 2A and 2B, the clamp mechanism being in a closed (disengaged) configuration, according to certain embodiments.

FIG. 14B illustrates a magnified perspective view of the clamp mechanism of FIG. 14A, the clamp mechanism being in a partially actuated configuration, according to certain embodiments.

FIG. 14C illustrates a magnified perspective view of the clamp mechanism of FIG. 14B, the clamp mechanism being in an open (engaged) configuration, according to certain embodiments.

FIG. 14D illustrates a magnified perspective view of the clamp mechanism of FIG. 14C, the clamp mechanism being in the open (engaged) configuration and in a distally displaced position, according to certain embodiments.

FIG. 14E illustrates a magnified perspective view of the clamp mechanism of FIG. 14D, the clamp mechanism being in the open (engaged) configuration and when a spring of the clamp mechanism is in a partially expanded state, according to certain embodiments.

FIG. 14F illustrates a magnified perspective view of the clamp mechanism of FIG. 14E, the clamp mechanism being in the open (engaged) configuration and when the spring of the clamp mechanism is in a fully expanded state, according to certain embodiments.

FIG. 15A illustrates a side profile view of a clamp assembly comprising a plurality of clamp mechanisms actuated by a manual handle, the handle being in an initial proximal position relative to the fluidics subsystem, according to certain embodiments.

FIG. 15B illustrates a side profile view of a clamp assembly of FIG. 15A, the handle being in a partially actuated position to begin lifting a plate coupled to the clamp mechanisms, according to certain embodiments.

FIG. 15C illustrates a side profile view of a clamp assembly of FIG. 15B, the handle being in an advanced actuated position to further lift the plate coupled to the clamp mechanisms, according to certain embodiments.

FIG. 15D illustrates a side profile view of a clamp assembly of FIG. 15C, the handle being in a fully actuated position to lift the plate coupled to the clamp mechanisms to a maximum height, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

The present disclosure relates generally to ophthalmic surgical consoles and ophthalmic surgical cassettes, and more specifically, to locking mechanisms for maintaining secure engagement between the surgical consoles and surgical cassettes, and methods of use thereof.

Certain embodiments disclosed herein provide clamp mechanisms for securely holding a small form factor surgical cassette to a fluidics module of an ophthalmic surgical console, thereby facilitating stable pressure sensor readings of fluids within the surgical cassette, while also maintaining engagement of surgical console irrigation and aspiration pump heads with the surgical cassette. In addition, certain embodiments disclosed herein enable maintained engagement of surgical console valve drives with the surgical cassette valves, as well as improved engagement of the fluidics module with the pump port seals of the surgical cassette.

Certain embodiments disclosed herein provide a clamping mechanism that minimizes physical stresses on the surgical cassette and ensures clamping forces are effectively applied to compress hub rollers of the surgical console against the surgical cassette for improved pump scaling and driving of fluid through the surgical cassette. In certain embodiments, the clamping mechanism is configured to apply clamping forces evenly to a plurality of clamping points of the surgical cassette. In certain embodiments, the clamping mechanism comprises metal roller interfaces between a drive wheel 801 with motion pin and a motion plate to eliminate sliding friction, thereby allowing higher clamping forces to be used and contributing to a longer use life of the clamping mechanism.

In certain embodiments, a clamping mechanism is provided which comprises clamp arm assemblies that are configured to open in a scissor-like fashion to engage the surgical cassette evenly above and below a center line of one or more pumps and further provide a small form factor. For example, in certain embodiments, the clamp arm assemblies comprise a multi-bar linkage system that provides high clamping forces with an over-center position and that clamps the surgical cassette in eight locations while being actuated by one single clamp motor, thereby ensuring that the clamping mechanism maintains a small and thin form factor. In certain embodiments, the clamp assemblies are disposed at an outer peripheral portion of the fluidics module and surgical cassette, thereby leaving sufficient room for the various components of the surgical cassette and the surgical console, including but not limited to pump heads, valve drives, pressure sensors, a barcode reader, fluid level sensors, and a venturi or fluid port.

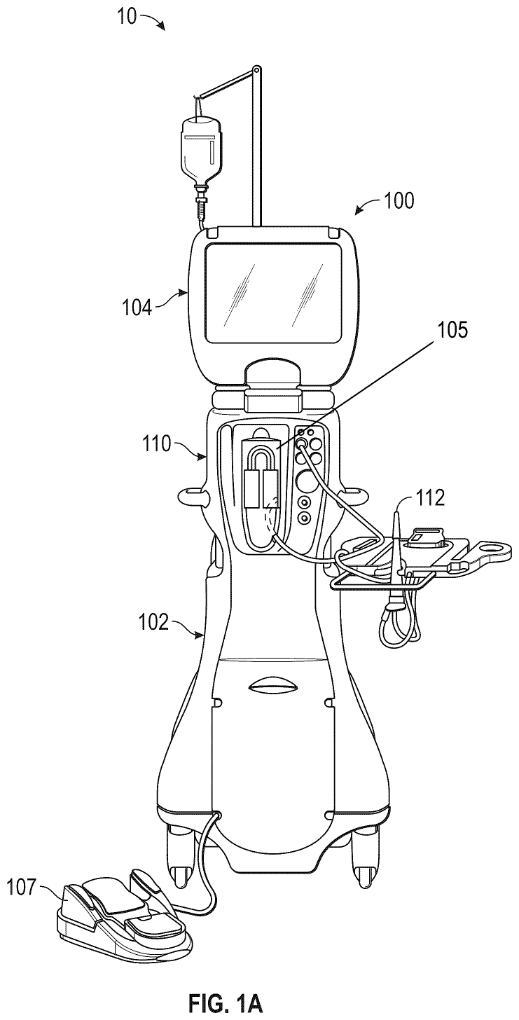

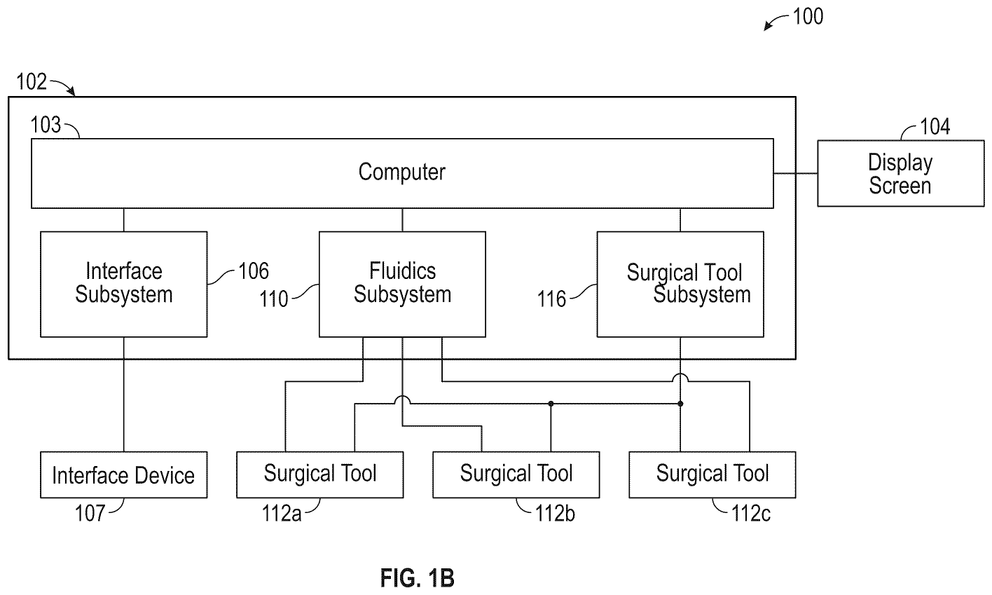

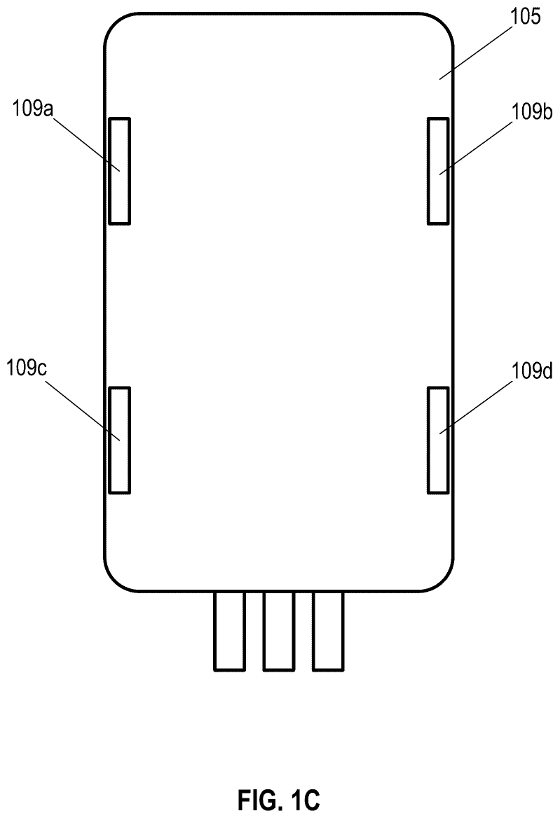

Turning now to FIG. 1A, an example of an ophthalmic surgical system 10 that may be used to perform ophthalmic procedures on an eye is illustrated, according to certain embodiments. In the illustrated embodiments, system 10 includes console 100 (also referred to as a “surgical console”), a housing 102, a display screen 104, an interface device 107 (e.g., a foot pedal), a fluidics subsystem 110, a surgical cassette 105, and a surgical tool 112, coupled as shown and described in more detail with reference to FIG. 1B. Note that the console 100 is merely an exemplary depiction of a console for illustration purposes, and that the embodiments described herein are applicable to a variety of consoles that may look or function similarly or differently in relation to console 100. FIG. 1C illustrates cassette slots 109a-d on surgical cassette 105 for engagement with corresponding hooks of the clamp assembly as discussed below, according to certain embodiments. Other features (e.g., pump elastomers, sensors, valves, etc.) of the cassette 105 are not shown.

FIG. 1B illustrates example subsystems of console 100 of ophthalmic surgical system 10 of FIG. 1A, according to certain embodiments. Console 100 includes housing 102, which accommodates a computer 103 (with an associated display screen 104) and subsystems 106, 110, and 116, which support interface device 107 and surgical tools 112 (112a-c). An interface device 107 receives input to surgical system 10, sends output from system 10, and/or processes the input and/or output. Examples of an interface device 107 include a foot pedal, a manual hand input device (e.g., a keyboard or handheld device), and a display. Interface subsystem 106 receives input from and/or sends output to interface device 107.

Surgical tool 112 may be any suitable ophthalmic surgical instrument, e.g., an ultrasonically driven phacoemulsification (phaco) probe, a laser probe, an irrigating cannula, a vitrectomy probe, or the like. Fluidics subsystem 110 provides fluid control for one or more surgical tools 112 (112a-c). For example, fluidics subsystem 110 may manage fluid for an irrigating cannula. Surgical tool subsystem 116 supports one or more surgical tools 112. For example, surgical tool subsystem 116 may manage ultrasonic oscillation for a phaco probe, provide laser energy to a laser probe, control operation of an irrigating cannula, and/or manage features of a vitrectomy probe.

Computer 103 controls operation of ophthalmic surgical system 10. In certain embodiments, computer 103 includes a controller, with associated processor and memory, which sends instructions to components of system 10 to control system 10. A display screen 104 shows data provided by computer 103.

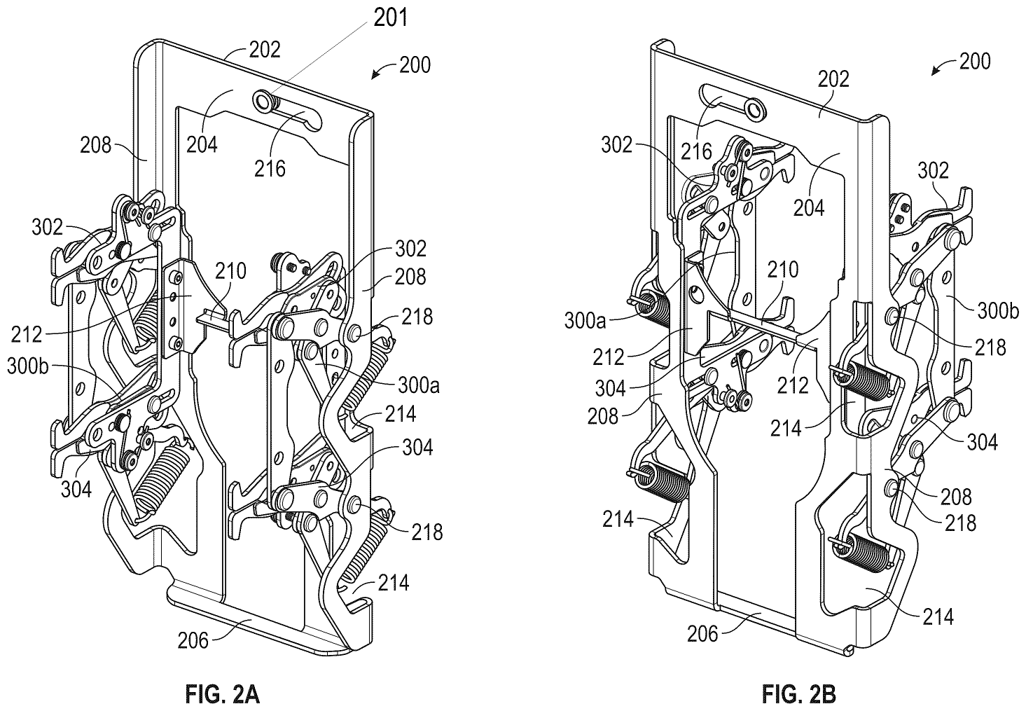

FIGS. 2A and 2B illustrate frontal and rear perspective views, respectively, of an example clamp assembly 200 used to couple and maintain a surgical cassette 105 to the console 100 and in particular to the fluidics subsystem 110. The clamp assembly 200 comprises a motion plate 202 comprising a top portion 204 joined to a bottom portion 206 by a pair of side portions 208. In certain embodiments, the top portion 204 comprises a motion slot 216 defined therein. In some embodiments, the motion slot 216 may include a grooved bushing 201 that is configured to engage a motion pin. In certain other embodiments, coupled to each of the side portions 208 is a first clamp sub-assembly 300a and a second clamp sub-assembly 300b. Each of the first and second clamp sub-assemblies 300a, 300b in turn comprise an upper clamp mechanism 302 and a lower clamp mechanism 304.

As shown in FIG. 2B, in certain embodiments, the clamp assembly 200 further comprises a bracket 212 with a brace 210 horizontally disposed between the pair of clamp sub-assemblies 300a and 300b and coupled to a guide bracket 328 at either lateral end to a corresponding one of the clamp sub-assemblies 300a and 300b. The clamp sub-assemblies 300a and 300b are horizontally disposed between the pair of side portions 208 of the motion plate 202 with the corresponding motion levers 306 of the clamp sub-assemblies 300a and 300b being coupled to the corresponding side portions 208 of the motion plate 202. Each of the side portions 208 further comprises a plurality of apertures 214 defined in their corresponding rear surfaces which are configured to accommodate a portion of the upper clamp mechanism 302 and a portion of the lower clamp mechanism 304, respectively. While the clamp assembly 200 is seen as comprising two clamp sub-assemblies 300a, 300b in FIGS. 2A and 2B, in certain embodiments, the clamp assembly 200 may comprise fewer or additional clamp sub-assemblies 300a, 300b other from what is explicitly shown, with each clamp sub-assembly 300a, 300b in turn comprising fewer or additional clamp mechanisms 302, 304 other from what is explicitly shown as well.

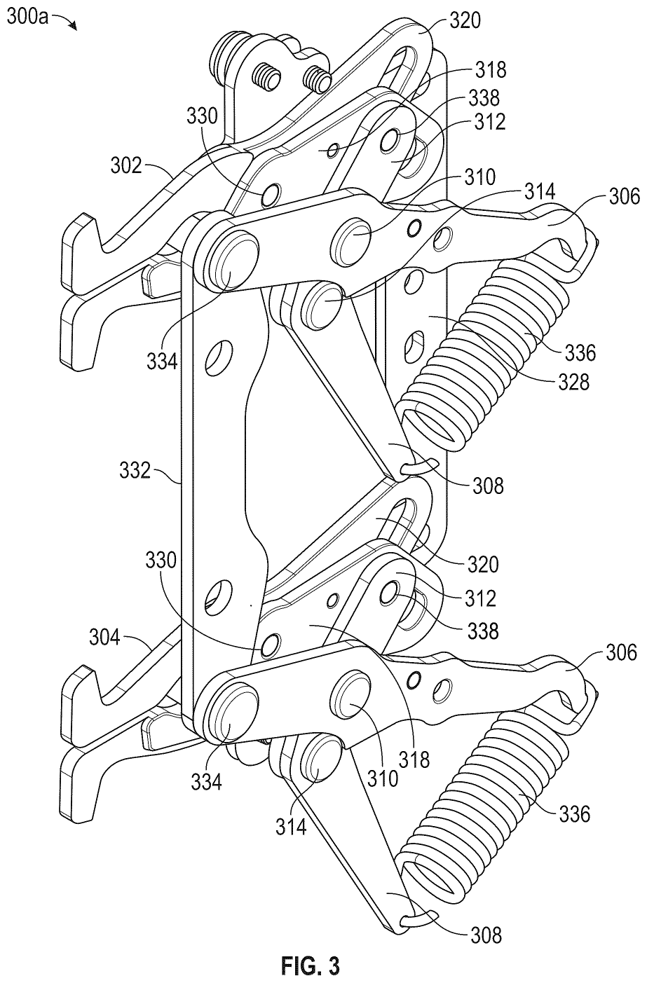

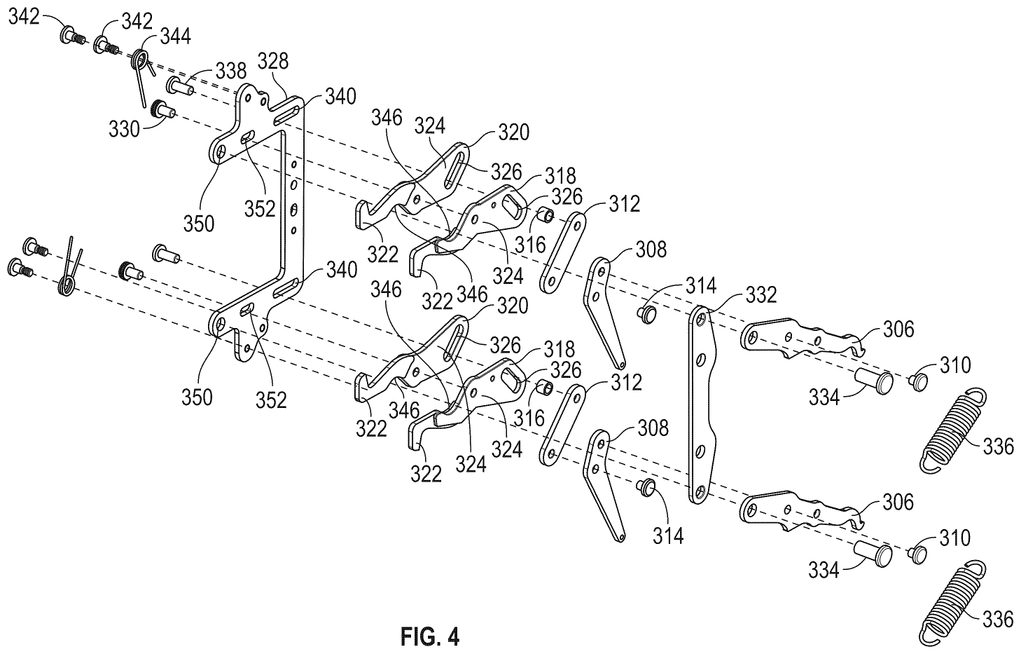

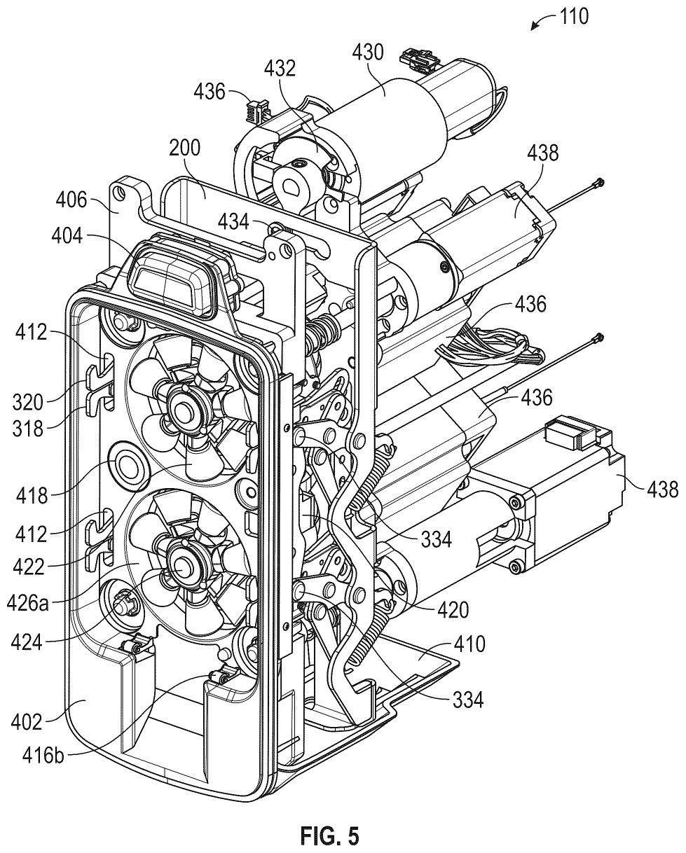

Turning now to FIGS. 3 and 4, in certain embodiments, each clamp sub-assembly 300 comprises an upper clamp mechanism 302 and a lower clamp mechanism 304. Each clamp mechanism 302, 304 comprises a first lever 306 coupled to a second lever 308 via a lever pin 310. Also coupled to the second lever 308 is a linkage 312 by a linkage pin 314 that is disposed through a first lateral end of the linkage 312. A pin 338 is disposed through the distal slot of the guide 328, a spacer 316, and a second lateral end of linkage 312. The spacer 316 is in turn is disposed through a first arm 318, and a second arm 320, while disposed between the guide 328 and the linkage 312. The first arm 318 and the second arm 320 are jointly coupled to the proximal slot of the guide 328 by a guide pin 330 which is disposed through a center portion of each arm 318, 320. A fixed or base pin 334 is further disposed through the first lever 306 and coupled to a base 332. Furthermore, the base pin 334 is disposed through an aperture defined by a cutout 346 that is defined in each of the first and second arms 318, 320 (to clear the first and second arms 318, 320). The distal end of the base pin 334 terminates or is coupled to the guide 328 which thereby forms a rigid assembly of the base pin 334, the base 332, and the guide 328 while capturing the first lever 306 and allowing it to rotate. A clamp spring 336 is further coupled to each proximal end of the first lever 306 and second lever 308, thereby providing a biasing force between the base pin 334 and the linkage pin 314 such that when the clamp mechanism is fully actuated, the distance between these pins 334 and 314 is compressed and the biasing force provides the clamping force. As used herein, the term “distal” refers to the area or region that is closer to the back or rear of the fluidics subsystem 110, for example the area comprising the valve motors 438 and pump motors 436 as seen in FIG. 5, while the term “proximal” refers the area or region that is closer to the front of the fluidics subsystem 110, for example the area comprising the bezel 402 and the face plate 406 as also seen in FIG. 5. As shown in FIG. 4, in certain embodiments, both the first arm 318 and the second arm 320 comprise a body 324 with a hook 322 disposed on a proximal end of the body 324 and an angled track 326 defined in a distal end of the body 324. In certain embodiments, the first arm 318 and the second arm 320 are similar except the pivot hole involves clearance on one and interference on the other). In certain embodiments, the hook 322 of the first arm 318 is orientated in a downward direction, while the hook 322 of the second arm 320 is orientated in an upward direction. Similarly, the angled track 326 of the first arm 318, when viewed from a side profile, is angled in a downward direction, while the angled track 326 of the second arm 320, when viewed from a side profile, is angled in an upward direction. In certain embodiments, the spacer 316 is disposed or inserted through each respective angled track 326 of the first arm 318 and the second arm 320. A clamp pin 338 disposed through a guide slot 340 defined within the guide 328 is coupled to or inserted into the spacer 316 and is coupled to the linkage 312 at one lateral end, thereby further coupling the linkage 312, the first arm 318, and the second arm 320 to the guide 328. A torsion spring 344 is coupled to an inward facing surface of the guide 328 by a plurality of torsion spring pins 342. The torsion spring 344 is further coupled to the guide pin 330 which further assists with the movement of the first and second arms 318, 320 as discussed in further detail below.

In certain embodiments, the guide 328 comprises a mount hole 350 for the base pin 334, a first guide slot or channel 352, and a second guide slot or channel 340. Two opposed bodies 324 are connected near the center with a guide pin 330 so that they can rotate about the guide pin 330. The guide pin 330 is disposed through the first guide channel 352 of the guide 328 to constrain a motion of the guide pin 330 from front-to-back and back-to-front. The torsion spring 344 is configured to apply a return force to the guide pin 330 acting from the guide 328. The clamp pin 338 is disposed through the angled tracks 326 at the distal end of each of the bodies 324 and through the secondary guide channel 340 of the guide 328 so that the clamp pin 338 is constrained to move only in the distal and proximal directions. The clamp pin 338 is mounted to the linkage 312 near the back of the assembly which is configured to move the clamp pin 338. Additionally, the spacer 316 may be used to fit over the clamp pin 338 and through the respective angled tracks 326 of each body 324 and between the guide 328 and the linkage 312 to ensure space and prevent binding.

Movement of the clamp pin 338 from front-to-back, or from a distal position near the front of the surgical console 100 to a proximal position near the back of the surgical console 100, pushes against the angled tracks 326 of each body 324 and rotates each body 324, which “expands” or “contracts” the distance between the hooks 322 and thus engages or disengages with the cassette 105. The linkage 312 is mounted at an opposing end to a linkage arm assembly defined by the first lever 306, the second lever 308, and the lever pin 310, with a linkage pin 314 disposed therein to allow relative rotation. The linkage arm assembly defined by the first lever 306, the second lever 308, and the lever pin 310 is mounted to the base pin 334 near the front of the assembly. The linkage arm assembly 306, 308, 310, can extend or compress between its two connecting points, specifically at base pin 334 and linkage pin 314, and provides an expansion force by use of the clamp spring 336. Upward movement of the lever pin 310 acts to rotate the linkages and displace the clamp pin 338 distally which in turn expands the hooks 322 of each body 324. Alternatively, a plate pin 218 may be added at another location on the linkage 312 or the linkage arm assembly 306, 308, 310, and used to actuate the clamp mechanism 302, 304.

In certain embodiments, mounting of the clamp mechanism 302, 304 is accomplished through mounting holes for screws in the base 332. The base 332 is coupled to the base pin 334, which in turn secures the guide 328. The base 332 can be arranged so as to be opposite of the guide 328 from each of the two bodies 324 to provide additional space for other modules (e.g., fluidics module) and/or cassette features. In this arrangement, the base pin 334 traverses through a recess in each body 324 to connect the guide 328 to the base 332 and provide a supporting connecting point for the linkage arm assembly 306, 308, 310.

In certain embodiments, the upper clamp mechanism 302 and the lower clamp mechanism 304 of each of the first and second clamp sub-assemblies 300a, 300b are substantially identical. Specifically, both the upper clamp mechanism 302 and the lower clamp mechanism 304 each comprise a first and second lever 306, 308, a linkage 312, and a first and second arm 318, 320 in the configuration detailed above. In certain embodiments, each of the upper clamp mechanism 302 and the lower clamp mechanism 304 are coupled to a common base 332 and to a common guide 328. The base 332 and the guide 328 each comprise symmetrical or vertical mirror image configurations thereby allowing both the upper clamp mechanism 302 and the lower clamp mechanism 304 to be coupled to the upper and lower portions thereof in substantially similar manners.

Returning to FIGS. 2A and 2B, in certain embodiments, the first clamp sub-assembly 300a and the second clamp sub-assembly 300b are also similar. In some embodiments, the assembly method may use similar non-mirrored parts of each other, with the first lever 306 of each respective clamp mechanism 302, 304 seen as being disposed at the outer most edge or periphery of each clamp mechanism 302, 304 when the clamp assembly 200 is viewed from a frontal angle. The first lever 306 of each respective clamp mechanism 302, 304 is coupled to a corresponding side portion 208 of the motion plate 202 via the plate pin 218.

In certain embodiments, the clamp assembly 200 is incorporated into the fluidics subsystem 110 of the console 100, as shown in FIGS. 5-7. Looking at FIG. 5, the clamp assembly 200 is disposed on a proximal or frontal portion of the fluidics subsystem 110, near a frontal surface of the console 100. In certain embodiments, the clamp assembly 200 is disposed behind a face plate 406 of the fluidics subsystem 110 and over a drain pan 410 coupled to the face plate 406. The clamp assembly 200 is coupled to the face plate 406 by a shoulder screw which is inserted through each of the upper and lower clamp mechanisms 302, 304 and terminates within a corresponding aperture (e.g., a threaded hole) within a side portion or edge of the face plate 406. The face plate 406 comprises a plurality of slots 412 defined through its surface, which in certain embodiments, are symmetrically defined vertically and horizontally about the face plate 406, and in certain embodiments, defined on either side of a pair of substantially circular apertures 444 also defined in the face plate 406. When the fluidics subsystem 110 is assembled, the clamp assembly 200 and face plate 406 are aligned with one another so that the hooks 322 and distal portions of the first arm 318 and second arm 320 of the upper and lower clamp mechanisms 302, 304 are disposed through and extend out of a corresponding slot 412. Each slot 412 is large enough to permit relative vertical movement of the hooks 322, as well as a degree of horizontal movement therein.

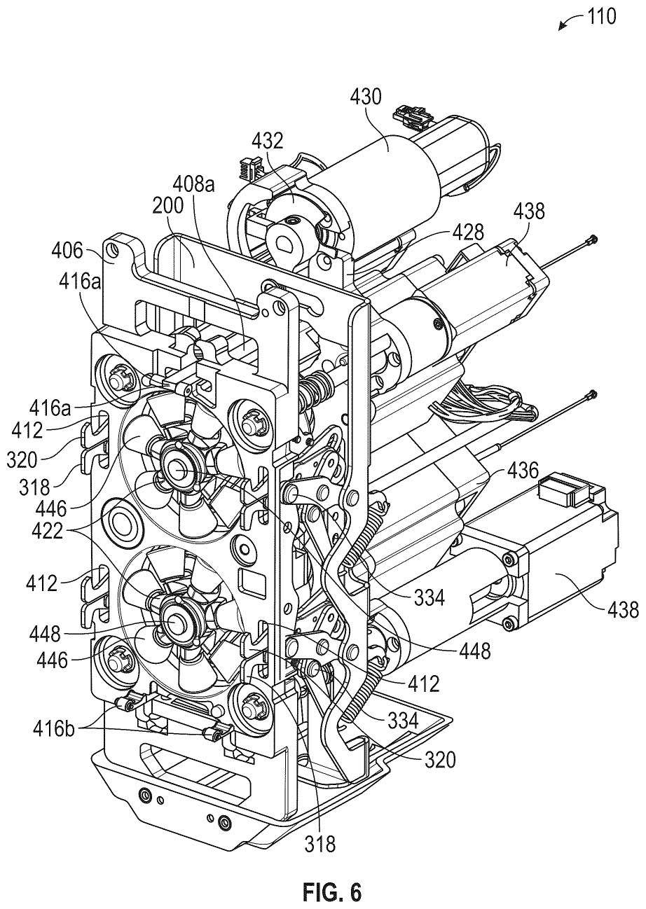

In certain embodiments, the face plate 406 further comprises and accommodates within its apertures 444 a pair of hub roller assemblies 422 disposed therein, wherein hub rollers 446 of each hub roller assembly 422 are arranged in a circular fashion around a hub 448. A pair of first fluid shields 426a and a pair of second fluid shields 426b surround the hub roller assemblies 422 and ensure that any fluids do not leak into any adjacent components. A pressure sensor 418 and a camera 420 are further disposed through the face plate 406 for monitoring a surgical cassette 105 when attached. In certain embodiments, the camera 420 is configured to read any barcodes on the surgical cassette 105. As shown in FIG. 6, an upper retaining arm 416a and a lower retaining arm 416b are disposed on a frontal surface of the face plate 406 for holding the surgical cassette 105 in place on the fluidics subsystem 110 as the clamp assembly 200 is actuated to securely lock the surgical cassette 105, as further detailed below.

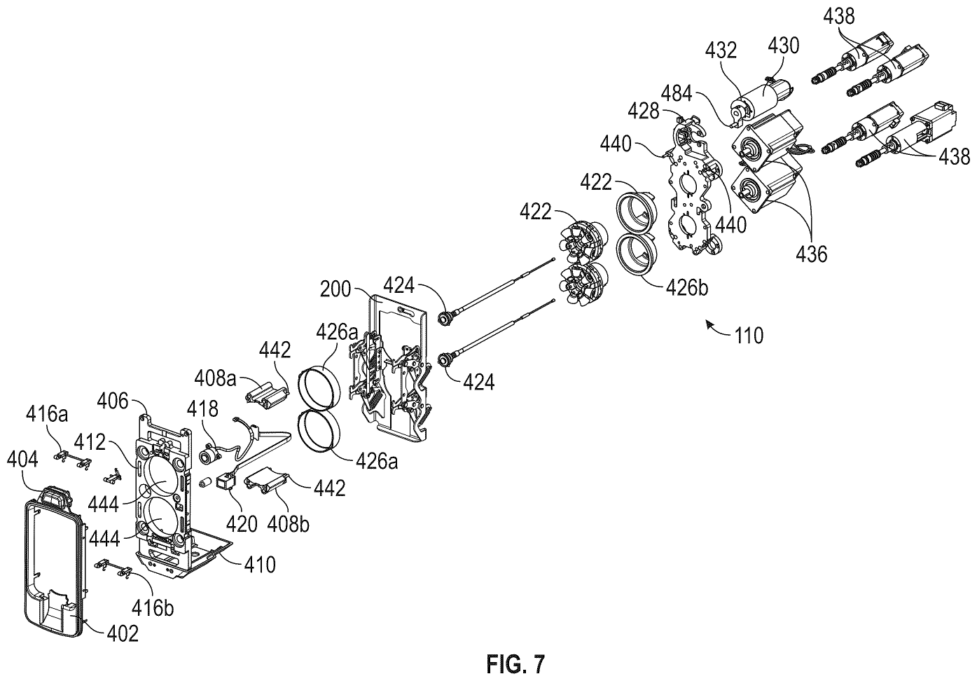

In certain embodiments, a bezel 402 is coupled to the frontal surface of the face plate 406 as shown in FIGS. 5 and 7. The bezel 402 has an opening or aperture defined therein which is large enough to dispose a surgical cassette 105 there through in order to removably couple to the exposed face plate 406. A button 404 disposed on a top portion of the bezel 402 may be used to actuate the clamp assembly 200 as is further detailed below.

In certain embodiments, a first spacer 408a and a second spacer 408b are coupled at one lateral end to a top portion or a bottom portion of the face plate 406, respectively. The first and second spacers 408a, 480b are also coupled to the clamp assembly 200 at another lateral end and serve as guides for the clamp assembly 200 with each of the spacers 408a, 408b providing a complimentary shape for nesting or forming a tight fit with the top portion 204 and bottom portion 206 of the motion plate 202, respectively. In certain embodiments, each of the spacers 408a, 408b comprises a plurality of spacer pegs 442, which further interact with motion plate 202 of the clamp assembly 200 to limit its horizontal movement relative to the rest of the fluidics subsystem 110.

In certain embodiments, a motor plate 428 is disposed distally behind the clamp assembly 200. The motor plate 428 comprises a plurality of motor plate pegs 440, which interact with the motion plate 202 of the clamp assembly 200 to further limit the relative horizontal movement of the clamp assembly 200 while being used. A motion plate motor 430 and a plurality of pump motors 436 are coupled to the motor plate 428. In certain embodiments, the motion plate motor 430 comprises a rotating drive wheel 801 with motion pin 434 that is inserted through the motion slot 216 (e.g., through a grooved bushing 201) in the top portion 204 of the motion plate 202. In certain embodiments, a flag 432 (or other indicator) is disposed on the drive wheel 801 with motion pin 434, which operates in conjunction with an optical sensor to determine the current state or status of the motion pin 434 and/or the motion plate motor 430. The pump motors 436 are coupled to the motor plate 428 and extend though the interior of the motion plate 202 of the clamp assembly 200 so as to engage each of the hub roller assemblies 422, which in turn serve to circulate or move fluid through the surgical cassette 105 when attached. Disposed at or near the hub 448 of each hub roller assembly 422 is an additional pressure sensor 424 for monitoring the pressure within the cassette 105. In certain embodiments, a plurality of valve motors 308 are also disposed on either side of the motor plate 428 and likewise extend or are disposed through the motion plate 202. The distal ends of the valve motors 438 also extend through the face plate 406 and serve to actuate a corresponding plurality of valves that are disposed within the cassette 105.

In certain embodiments, a surgical cassette 105 is first inserted into the fluidics subsystem 110 by orientating it over the face plate 406 and then disposing a plurality of slots or holes defined in a rear surface of the cassette 105 over the corresponding plurality of hooks 322 extending from the surface of the face plate 406 while the clamp assembly 200 is in a closed configuration. In certain embodiments, the cassette 105 is held temporarily in place by the upper and lower retaining arms 416a, 416b.

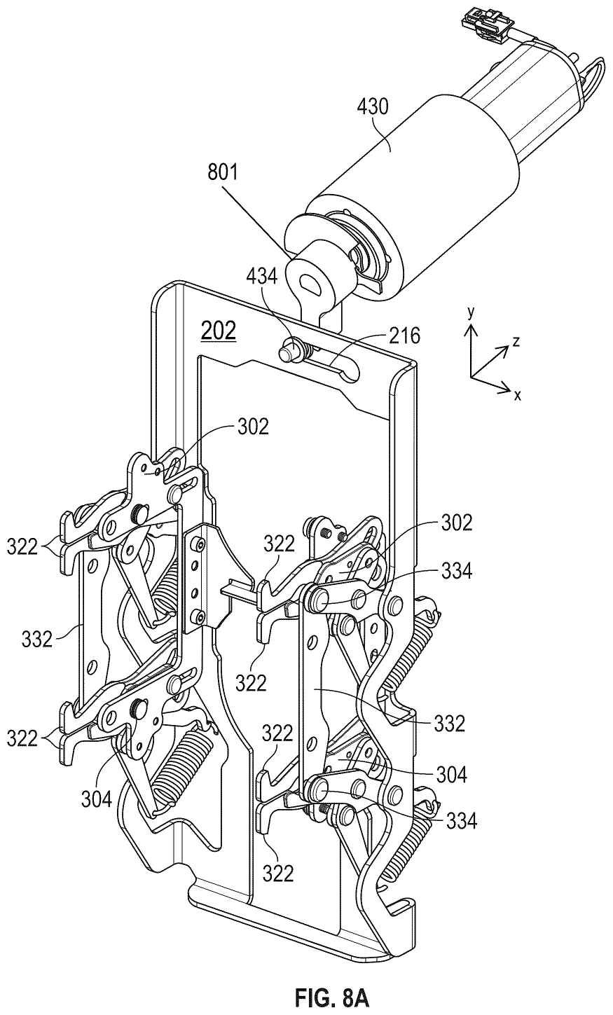

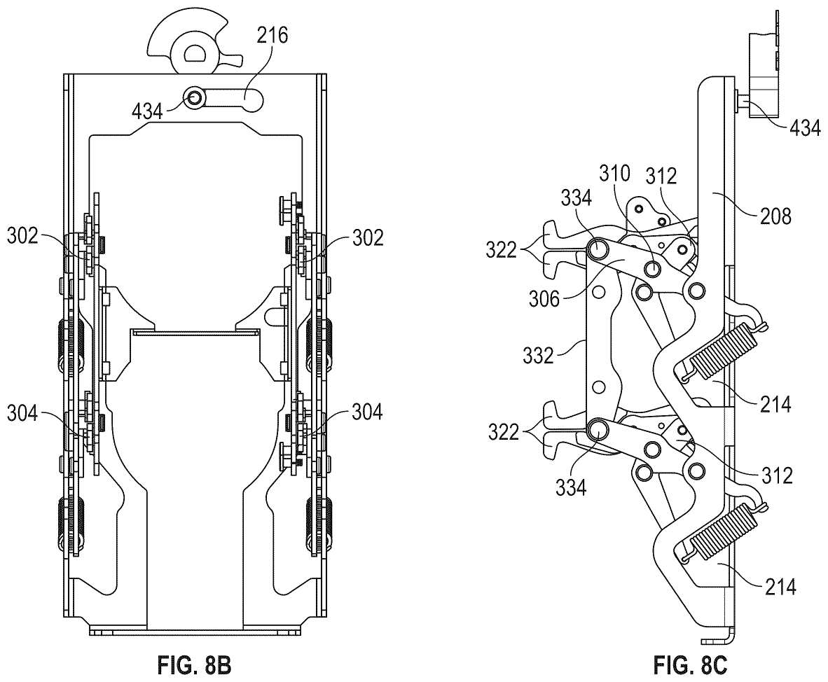

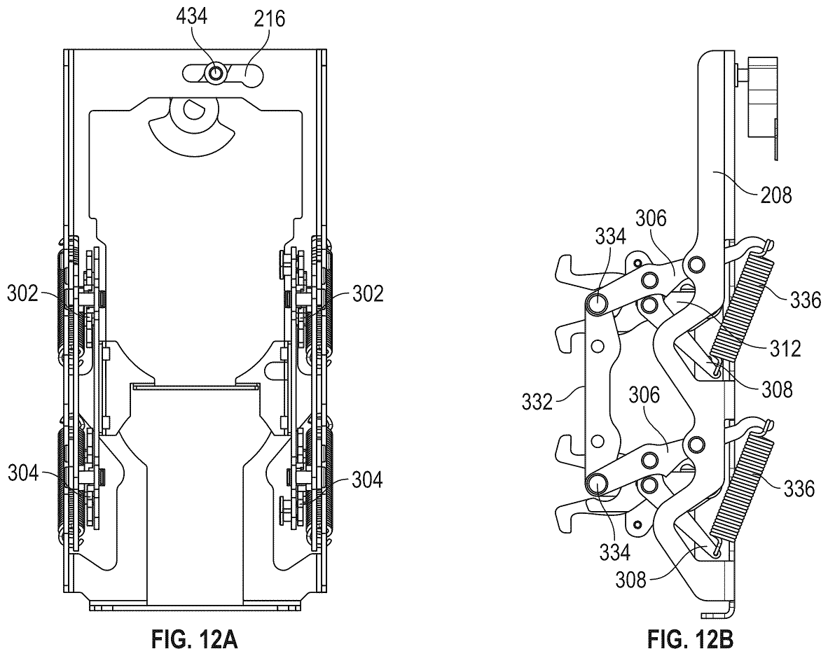

FIGS. 8A-13B illustrate an example of how the clamp assembly 200 is actuated so as to couple the surgical cassette 105 to the fluidics subsystem 110. FIGS. 8A-8C show the clamp assembly 200 in an initial, or closed (disengaged), configuration with the upper and lower clamp mechanisms 302, 304 of each clamp sub-assembly 300a, 300b in the “closed” (disengaged) position, namely wherein the first and second arms 318, 320 are orientated so that the base pin 334 has clearance within the aperture defined between the first and second arms 318, 320 while the respective hooks 322 are adjacently disposed to each other and face opposing directions. When each clamp sub-assembly 300a, 300b is in the “closed” (disengaged) position, this allows the surgical cassette 105 to be disposed or slipped over the opposing hooks 322, namely by disposing a corresponding plurality of cavities or apertures (e.g., cassette slots 109a-b as seen in FIG. 1C) defined within the surgical cassette 105 over each of the hooks 322 so that the hooks 322 enter an internal portion of the surgical cassette 105. FIG. 8B also shows the motion pin 434 in an initial position at an extreme lateral end of the motion slot 216 defined in the motion plate 202.

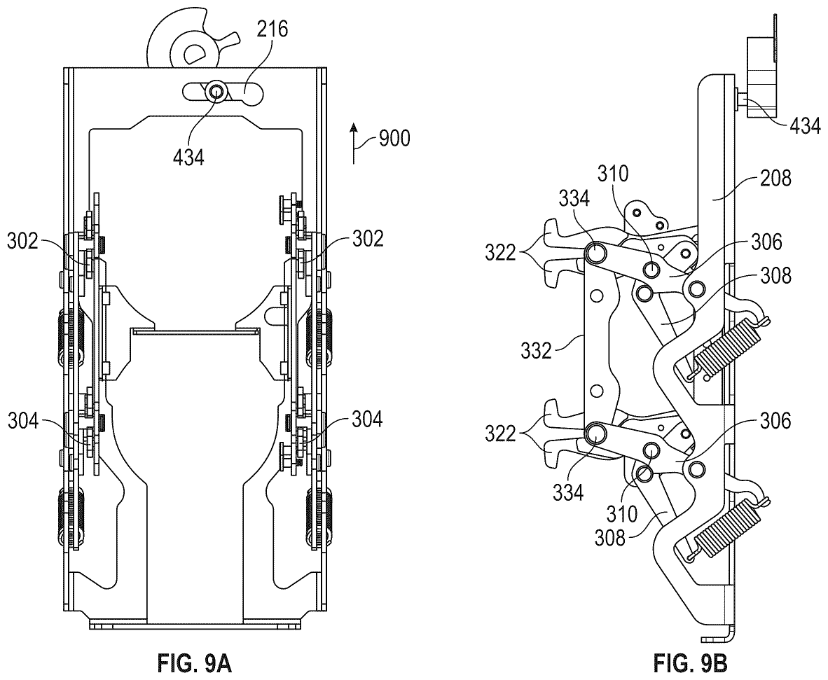

In certain embodiments, the clamp assembly 200 is activated upon cassette detection by optical transceivers on cassette detect PCBAs (printed circuit board assemblies) disposed on each side of the bezel 402 (FIGS. 5 and 7) which activates the motion plate motor 430. In some embodiments, the bezel 402 is transparent for infrared light and sensors may operate by interrupting the beam path and/or by reflection detection. When viewed from a frontal perspective, in certain embodiments, the motion plate motor 430 begins to rotate the motion pin 434 in a counterclockwise direction, which due to the substantially horizontal orientation of the motion slot 216, begins to move the motion pin 434 longitudinally through the length of the motion slot 216 as shown in FIG. 9A while simultaneously beginning to lift the motion plate 202 in the vertical direction, as indicated by arrow 900. As seen in FIG. 8A (respective to the displayed x-y-z axis), the upward/downward motion of the motion plate 202 may be substantially linear in the x-y plane and may sweep an arc in the y-z plane (side view). When viewed from the side perspective of FIG. 9B, as the motion plate 202 moves vertically upward, the side portion 208 moves the first lever 306 in the counterclockwise direction rotating about the base pin 334, while the second lever 308 rotates about the lever pin 310. In some embodiments, second lever does not rotate separately during this phase of motion. In some embodiments, 306, 308, and 338 form a virtual compressible linkage between 334 and 314 that also initially rotate counter clockwise about 334 so that 314 moves upwards rotating 312 clockwise and sliding 338 inwards which expands the hooks for phase 1 motion until 338 reaches the end of the slots. In some embodiments, as 338 continues inward motion, the clamp arms are moved inward pulling the cassette into the module for the next phase of motion until the cassette contacts into the faceplate and 338 can no longer move inward. In some embodiments, as 306 continues to rotate counterclockwise, the virtual linkage between 334 and 314 compresses while the extension spring expands to apply an increasing clamping force on the cassette for the third phase of motion until the actuation ceases.

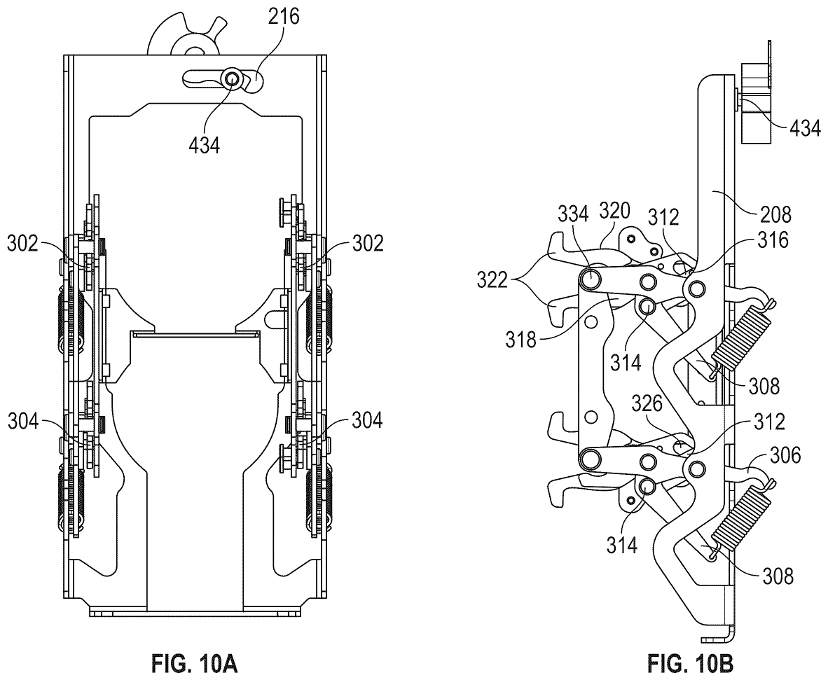

FIGS. 9A and 9B depict that as the drive wheel 801 with motion pin 434 continues to move the motion plate 202 upward by moving longitudinally through the motion slot 216, continued counterclockwise rotation of the first and second levers 306, 308 begins to rotate the linkage 312 in the opposing clockwise direction about the linkage pin 314 as viewed from the side perspective view of FIG. 10B. As the linkage 312 rotates, it moves the spacer 316 disposed in its lateral end in the distal direction which forces it to move longitudinally through the angled tracks 326 of both the first arm 318 and second arm 320. In certain embodiments, because the angled track 326 of the first arm 318 is orientated at a downward angle while the angled track 326 of the second arm 320 is orientated at an upward angle, the spacer 316 forces the first arm 318 to rotate counterclockwise and the second arm 320 to rotate clockwise about the guide pin 330 simultaneously, which in turn drives the hooks 322 further apart and thereby opens each upper clamp mechanism 302 and lower clamp mechanism 304. Because both the upper clamp mechanism 302 and the lower clamp mechanism 304 are also coupled to the base 332 which remains in a fixed position relative to the face plate 406 via the base pins 334, this ensures that all vertical movement of the motion plate 202 is transferred evenly and simultaneously to the upper and lower clamp mechanisms 302, 304, thereby ensuring that each opens up at the same time and at the same rate.

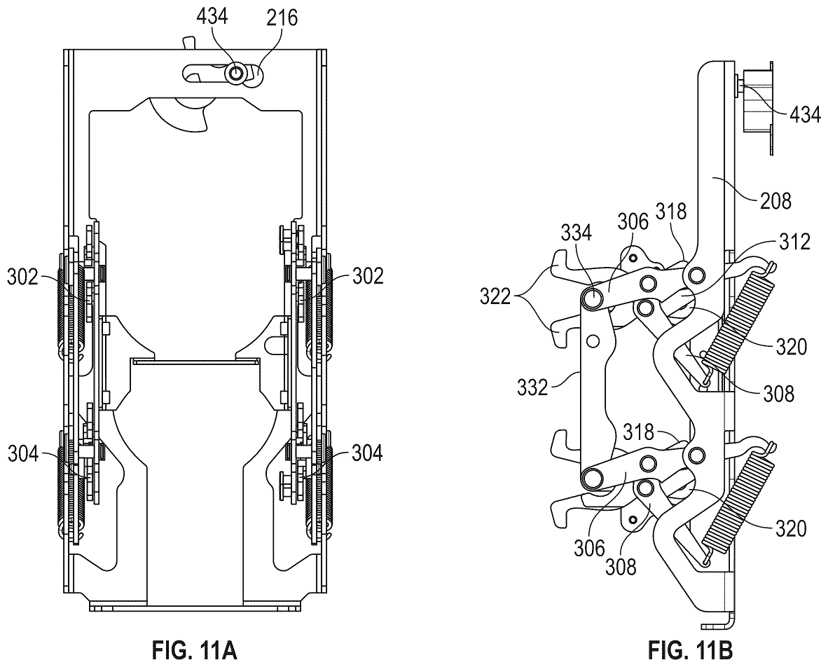

In FIGS. 11A and 11B, the motion plate motor 430 continues to drive the motion plate 202 vertically upward via the motion pin 434 until an “open” position is obtained, which, in certain embodiments, is when at least a portion of the angled tracks 326 of the first and second arms 318, 320 are in alignment with one another and with the spacer 316 being firmly disposed at a distal end of each angled track 326. Additionally, when in the “open” position, the hooks 322 of each clamp 302, 304 are disposed in maximum “open” positions as well, which, in certain embodiments, means that each of the respective hooks 322 are maximally displaced or separated from one another as shown in FIG. 11B.

In certain embodiments, the motion pin 434 continues to be rotated by the motion plate motor 430 to further drive the motion plate 202 upward, which due to the angle of rotation, then begins to move the motion pin 434 back in the opposing lateral direction across the motion slot 216.

In some embodiments, the spacer 316 disposed through the first and second arms 318, 320 can no longer move distally through the respective angled tracks 326, continued rotational movement of the first and second levers 306, 308 continues moving the pin 338 through the guide slot 340, pushing the spacer 316 against the end of the angled tracks 326 of the first and second arms 318, 320, moving the clamp mechanism 302 backwards, until the cassette 105 is pressed tight against the face plate 406 by the hooks 322 of the first and second arms 318,320. In certain other embodiments, after the backwards movement of the clamp mechanism 302 is prevented by the cassette 105 and faceplate 406, continued upward vertical movement of the motion plate 202 then begins to actuate the clamp springs 336 disposed between the first and second levers 306, 308, thereby providing a clamping force for each of the upper and lower clamp mechanisms 302, 304.

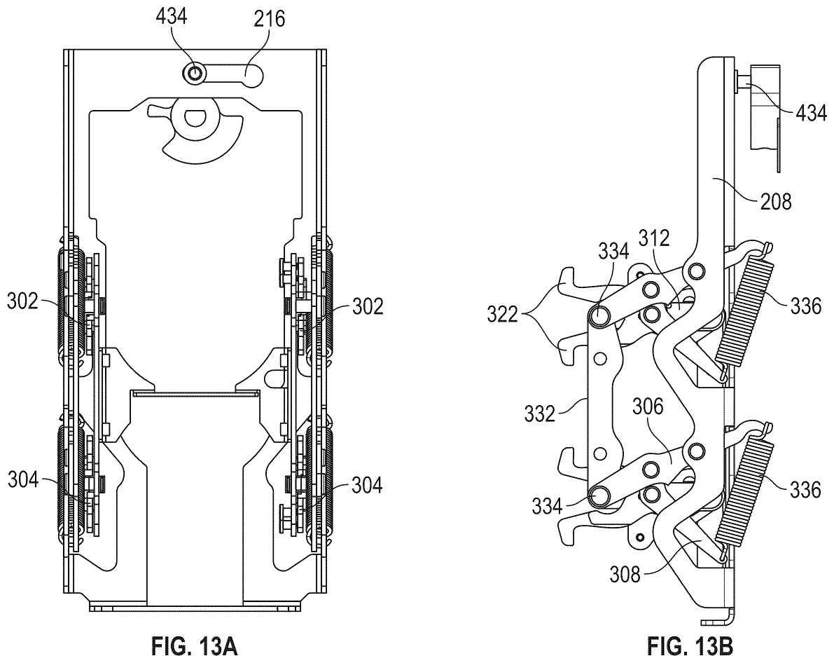

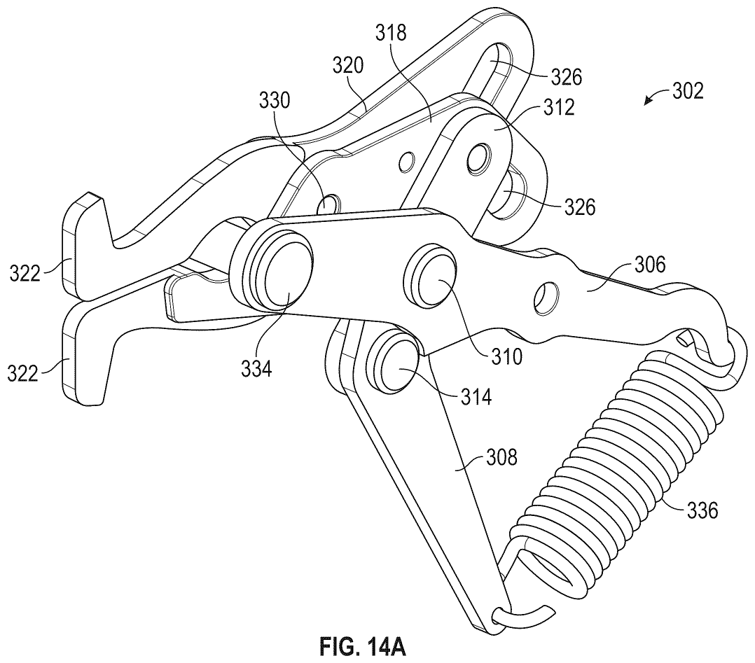

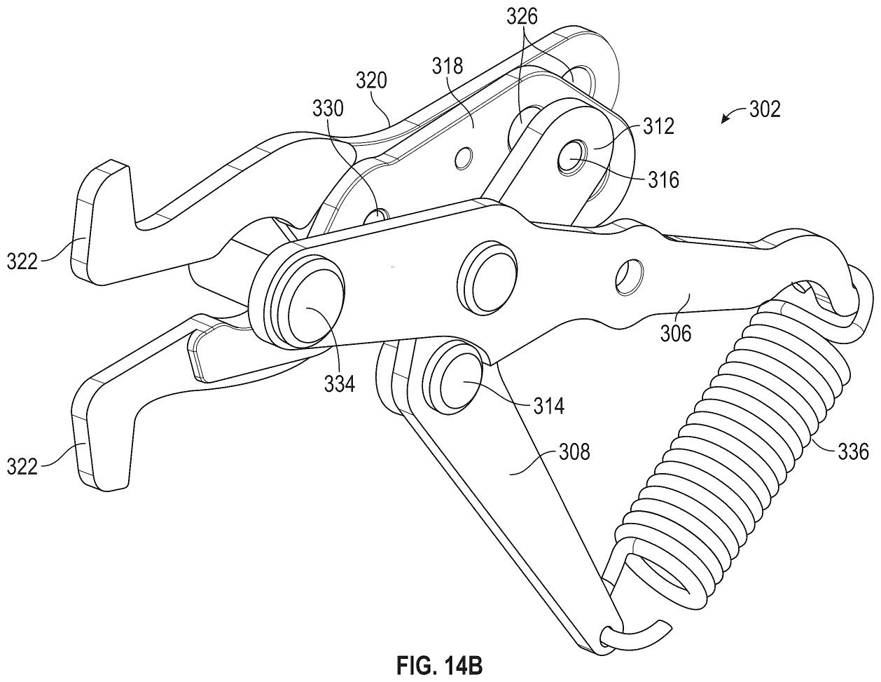

In certain embodiments of FIGS. 13A and 13B, the motion pin 434 has fully traversed the motion slot 216 and has come to a rest at one of its lateral ends. At this point, all vertical movement of the motion plate 202 stops and the upper and lower clamp mechanisms 302, 304 remain at a maximum open position with their respective hooks 322 disposed as their furthest position relative to each other.

In certain embodiments, as the hooks 322 of each of the upper and lower clamp mechanisms 302, 304 expand or open (engage) as discussed above, each of the hooks 322 expands within the slot or hole (e.g., cassette slots 109a-b as seen in FIG. 1C) defined within the surgical cassette 105 in which it is disposed and into a hollow interior or cavity defined therein. The angled ends of the hooks 322 ensure that a portion of each hook 322 extends through the surgical cassette 105 and remains there, thereby preventing or at least minimizing any distal movement of the surgical cassette 105 relative to the surgical console 100. The hooks 322 further pull the cassette 105 against the face plate 406, which ensures that engagement between the valve motors 438 and the surgical cassette valves are maintained while also ensuring that connections between the pressure sensors 418, 424 and the pump seals of the surgical cassette 105 and face plate 406 are firmly established.

In certain embodiments, a surgical cassette 105 is removed from the fluidics subsystem 110 by following the above process in reverse. Specifically, in certain embodiments, the user actuates the button 404 disposed on the bezel 402 which activates the motion plate motor 430 that in turn begins to rotate the motion pin 434 in a clockwise direction. The traversal of the motion pin 434 back and forth through the motion slot 216 in turn lowers or moves the motion plate 202 in the downward vertical direction in conjunction with the return bias provided by the clamp springs 336. As the first lever 306, second lever 308, and lever pin 310 rotate in the clockwise direction about the base pin 334, the linkage 312 rotates in the counterclockwise direction which expands the distance between pin 334 and pin 314 while decreasing the length of the clamp spring 336 until the first lever 306 contacts pin 314. After the first lever 306 rests against pin 314, the first lever 306, second lever 308, and lever pin 310 rotate together and pull the proximal end of linkage 12, which pulls pin 338 forward and thus pulls spacer 316 forward, allowing the torsion spring 344 to push the guide pin 330 with the first arm 318 and the second arm 320 forward until the guide pin 330 contacts the front end of the proximal slot in the guide 328. Subsequently, the spacer 316 is pulled forward in the proximal direction through the respective angled tracks 326 which in turn rotates each of the first and second arms 318, 320 accordingly and brings the hooks 322 back into close proximity with one another.

Because all upper and lower clamp mechanisms 302, 304 within the clamp assembly 200 are actuated by the movement of the motion plate 202, each set of hooks 322 is moved at the same time, thereby closing each clamp mechanism 302, 304 simultaneously. As the hooks 322 are being driven towards each other, the aperture defined by the cutouts 346 in the first and second arms 318, 320 once again encompass or are disposed around the base pin 334. With the upper and lower clamp mechanisms 302, 304 back in the closed configuration of FIGS. 8A-8C, the hooks 322 of each clamp mechanism 302, 304 are now close enough together so that when the user pulls the cassette 105 from the face plate 406, each pair of hooks 322 is easily removed from the corresponding slot or hole (e.g., cassette slots 109a-b as seen in FIG. 1C) defined within the cassette 105.

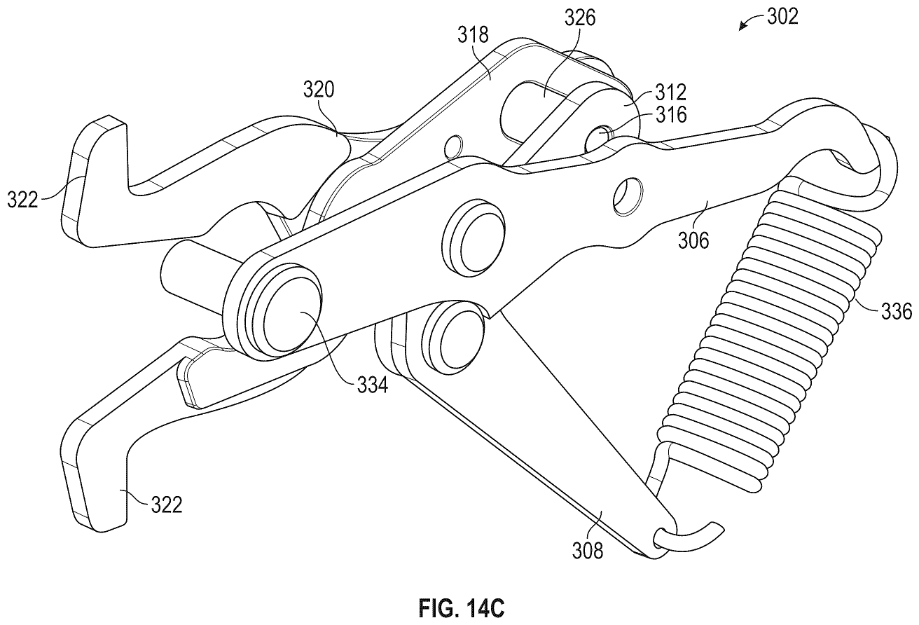

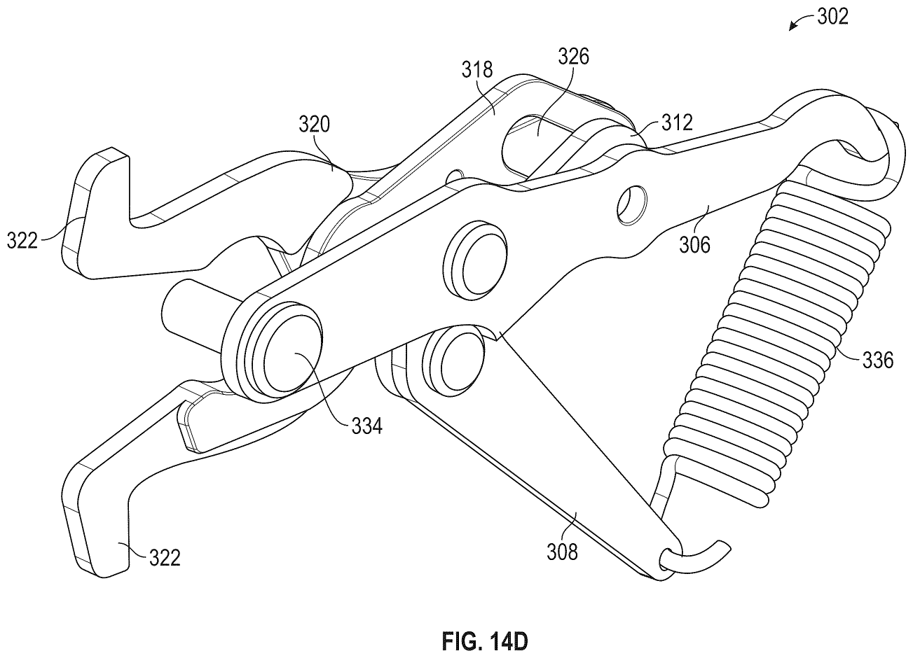

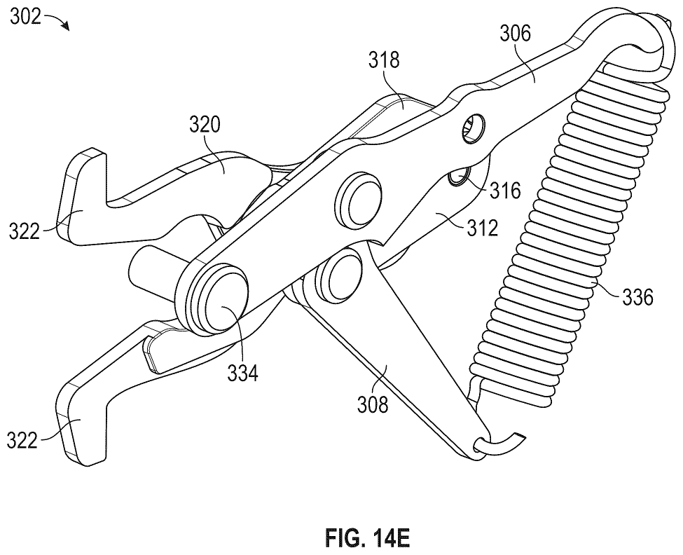

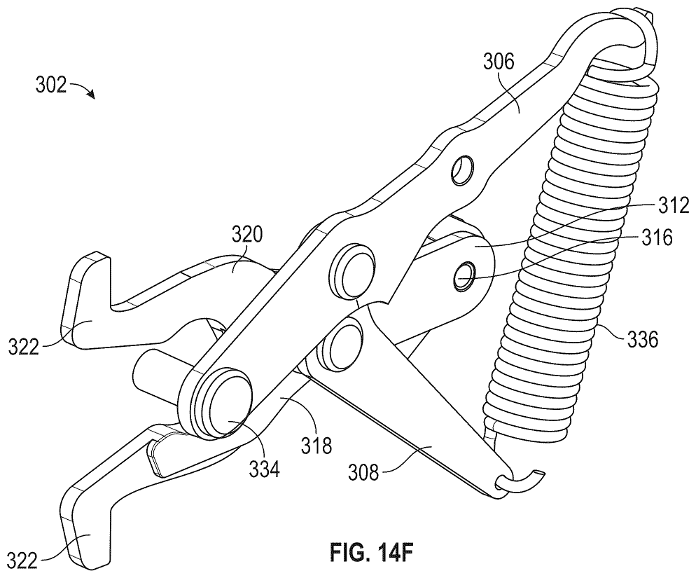

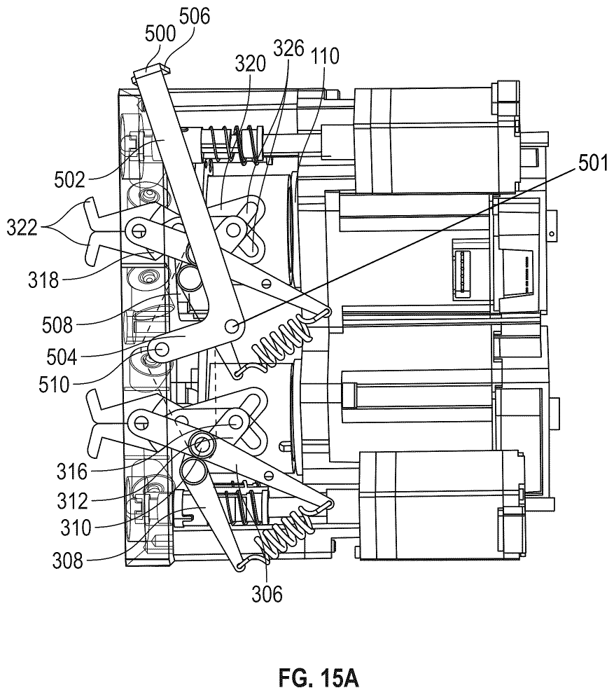

Greater detail of an example of how each upper and lower clamp mechanism 302, 304 functions is illustrated in FIGS. 14A-14F. While only one upper clamp mechanism 302 is shown, it is to be expressly understood that all clamp mechanisms 302, 304 within the clamp assembly 200 may operate in a similar fashion. Additionally, it should be noted that the base 332 and the motion plate 202 have been removed from the figures so as to provide clear and unobstructed views of the clamp mechanism 302.

FIG. 14A show the clamp mechanism 302 in an initial or closed configuration, namely wherein the first and second arms 318, 320 are orientated so that the respective hooks 322 are adjacently disposed to each other and face opposing directions.

In certain embodiments, after the clamp assembly 200 is activated and the motion plate 202 moves vertically upward, the side portion 208 pulls the first lever 306 and the second lever 308 upward which then begin to rotate in the counterclockwise direction, rotating about the base pin 334. As shown in FIGS. 14B-14D. The clamp spring 336 holds the first lever 306 and second lever 308 together, keeping the first lever 306 in contact with the linkage pin 314, they rotate along with lever pin 310 and move linkage pin 314, and in turn rotate linkage 312 in the clockwise direction. As the linkage 312 rotates, it pushes the spacer 316 disposed in its lateral end in the distal direction which forces it to move longitudinally through the angled track 326 of both the first arm 318 and second arm 320. In certain embodiments, because the angled track 326 of the first arm 318 is orientated at a downward angle while the angled track 326 of the second arm 320 is orientated at an upward angle, the spacer 316 forces the first arm 318 to rotate counterclockwise and the second arm 320 to rotate clockwise about the guide pin 330 simultaneously, which in turn drives the hooks 322 further apart and thereby opens the clamp mechanism 302.

The motion plate motor 430 continues to drive the motion plate 202 vertically upward until an open position is obtained which in certain embodiments is when at least a portion of the angled tracks 326 of the first and second arms 318, 320 are in alignment with one another with the spacer 316 being firmly disposed at a distal end of each angled track 326, as shown in FIG. 14C.

In certain embodiments, because the spacer 316 disposed through the first and second arms 318, 320 can no longer move distally through the respective angled tracks 326, continued rotational movement of the first and second levers 306, 308 continues moving the pin 338 through the guide slot 340, pushing the spacer 316 against the end of the angled tracks 326 of the first and second arms 318, 320, moving the clamp mechanism 302 backwards, until the cassette 105 is pressed tight against the face plate 406 by the hooks 322 of the first and second arms 318,320. In certain embodiments, after the backwards movement of the clamp mechanism 302 is prevented by the cassette 105 and faceplate 406, continued upward vertical movement of the motion plate 202 actuates the clamp springs 336 disposed between the first and second levers 306, 308, thereby providing a clamping force for the clamp mechanism 302 as shown in FIGS. 14D-14F. In embodiments of FIG. 14F, the clamp mechanism 302 remains at a maximum open position with its respective hooks 322 disposed as their furthest position relative to each other.

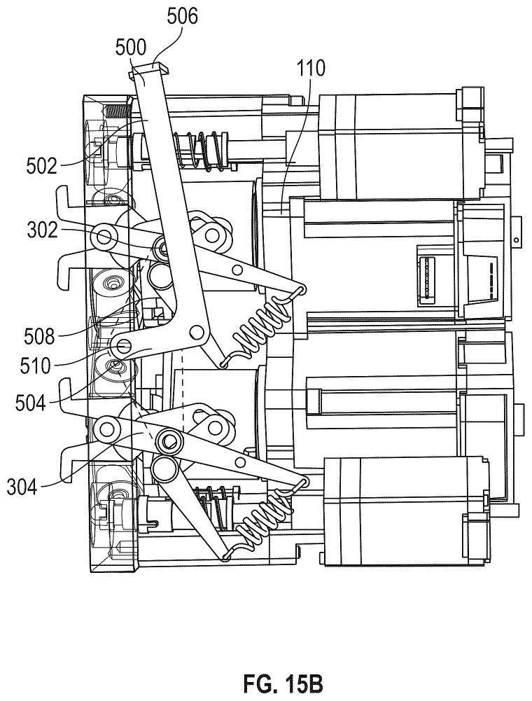

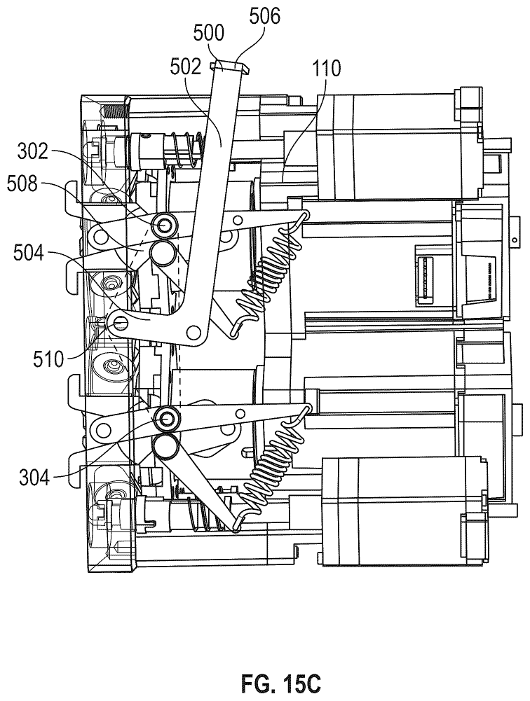

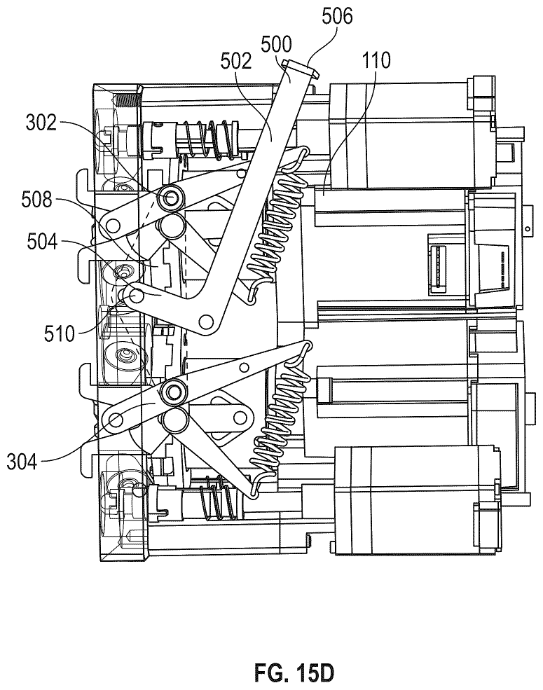

In certain other embodiments, the clamp assembly 200 is actuated by a hand or manual means, such as a handle 500 as shown in FIGS. 15A-15D. The handle 500 comprises a substantially “L” shaped side profile with a pair of elongated vertical portions 502 which each terminate in a horizontal portion 504. The handle 500 is disposed over the fluidics subsystem 110 with a vertical portion 502 disposed on either side thereof. A rail 506 joins the vertical portions 502 together and provides a handhold for the user to grip. The handle 500 is rotatably coupled near each junction of the elongated vertical portions 502 and the horizontal portions 504 to a frame bracket (not shown for clarity) on both sides of the fluidics sub-system 110 via a handle base pin 501. The frame bracket is affixed to the face plate 406 and remains stationary. Each horizontal portion 504 is rotatably coupled to a plate 508 via a handle lift pin 510. The plate 508 is in turn coupled to both the upper and lower clamp mechanism 302, 304 by the lever pin 310.

In certain embodiments, the handle 500 actuates the clamp assembly 200 in a similar manner as the motion plate 202 detailed above. For example, to open the clamp mechanisms 302, 304, a user grips the rail 506 and pushes it distally towards the back of the fluidics subsystem 110. As the handle 500 is pushed, it begins to rotate about the handle base pin 501 and lifts the handle lift pin 510 as seen FIGS. 15B-D, which in turn lifts the plate 508. The plate 508 then provides the same vertical movement as the motion plate 202 discussed above to rotate the first and second levers 306, 308, which in turn leads to the actuation of the first and second arms 318, 320 and the separation of each of the pair of hooks 322. The user may continue to push the handle 500 until a maximum position is reached as shown FIG. 15D when both the hooks 322 and the clamp springs 336 of each clamp mechanism 302, 304 are in the fully extended position.

Accordingly, improved clamping mechanisms for coupling ophthalmic surgical cassettes to an ophthalmic surgical console, and methods of use thereof, are provided herein.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

### EXAMPLE EMBODIMENTS

Embodiment 1: A clamp mechanism for engaging a surgical cassette with an ophthalmic surgical console, the clamp mechanism comprising: a plurality of bodies coupled together by a guide pin; a linkage coupled to the plurality of bodies by a clamp pin; and a linkage arm assembly coupled to the linkage, wherein the clamp pin is disposed through an angled track defined in each of the plurality of bodies, and wherein the clamp pin is configured to move in a proximal direction through the angled tracks of the plurality of bodies when the clamp mechanism is actuated, the proximal movement of the clamp pin forcing each of the plurality of bodies to rotate about the guide pin in opposing directions.

Embodiment 2: The clamp mechanism of Embodiment 1, further comprising a guide, wherein the base pin is disposed through a mount hole defined in the guide, wherein each of the plurality of bodies are configured to rotate about the guide pin and accommodate a base pin within a recess defined therein; wherein the guide pin is disposed through a first guide channel defined in the guide, and wherein the clamp pin is disposed through a second guide channel defined in the guide.

Embodiment 3: The clamp mechanism of Embodiment 2, wherein the guide pin is configured to move in a proximal direction through the first guide channel when the clamp mechanism is actuated, and wherein the clamp pin is configured to move in a proximal direction through the second guide channel when the clamp mechanism is actuated.

Embodiment 4: The clamp mechanism of Embodiment 2, further comprising a base coupled to the guide by the base pin.

Embodiment 5: The clamp mechanism of Embodiment 1, wherein the linkage arm assembly comprises: a first lever; a second lever; and a lever pin configured to couple the second lever to the first lever.

Embodiment 6: A fluidics subsystem for an ophthalmic surgical console, the fluidics subsystem comprising: a clamp assembly comprising a plurality of clamp mechanisms; a face plate coupled to a proximal side of the clamp assembly; and a motor plate coupled to a distal side of the clamp assembly, wherein a pair of hooks disposed on a proximal end of each of the plurality of clamp mechanisms are disposed through and extend from a corresponding plurality of slots defined through the face plate.

Embodiment 7: The fluidics subsystem of Embodiment 6, wherein the face plate comprises a plurality of hub rollers disposed therein and wherein the plurality of slots defined through the face plate are symmetrically defined on either lateral side of each of the plurality of hub rollers.

Embodiment 8: The fluidics subsystem of Embodiment 6, wherein the motor plate comprises a plurality of pegs configured to engage with the clamp assembly and limit a horizontal movement of the clamp assembly relative to the motor plate.

Embodiment 9: The fluidics subsystem of Embodiment 6, further comprising a motion plate motor coupled to the motor plate, the motion plate motor comprising a motion pin that is inserted into a motion slot defined in the clamp assembly.

Embodiment 10: The fluidics subsystem of Embodiment 6, further comprising a bezel coupled to the face plate, the bezel comprising a button configured to actuate the motion plate motor.

What is claimed is:

1. A clamp assembly for engaging a surgical cassette with an ophthalmic surgical console, the clamp assembly comprising:
a motion plate;
a plurality of clamp mechanisms each having a distal end and a proximal end, the distal end of each of the plurality of clamp mechanisms being coupled to the motion plate;
a plurality of bases, wherein each of the plurality of bases is coupled to proximal ends of at least two of the plurality of clamp mechanisms; and
a plurality of guides coupled to the plurality of clamp mechanisms,
wherein each of the plurality of clamp mechanisms comprises a pair of hooks disposed at each of their respective proximal ends.

2. The clamp assembly of claim 1, wherein the plurality of clamp mechanisms are horizontally and vertically symmetrically disposed on the motion plate.

3. The clamp assembly of claim 1, wherein the motion plate is configured to actuate each of the plurality of clamp mechanisms simultaneously.

4. The clamp assembly of claim 1, wherein each of the plurality of clamp mechanisms comprises:
a plurality of levers, wherein each lever of the plurality of levers has a clamp spring coupled to a distal end of the lever;
a linkage coupled to at least one of the plurality of levers; and
a plurality of arms coupled to the linkage,
wherein a distal end of at least one of the plurality of levers is coupled to the motion plate, and
wherein a proximal end of at least one of the plurality of levers is coupled to at least one of the plurality of bases;
wherein the pair of hooks of each of the plurality of clamp mechanisms are disposed on proximal ends of a pair of the plurality of arms.

5. The clamp assembly of claim 4, wherein each of the plurality of clamp mechanisms further comprises a spacer coupled to at least one of the plurality of guides,
wherein the spacer is disposed through an angled track defined in each of the plurality of arms, and
wherein the spacer is coupled to the linkage;
wherein the clamp assembly further comprises a movable clamp pin disposed through a guide slot defined in the at least one of the plurality of guides, wherein the clamp pin is coupled to the spacer.

6. The clamp assembly of claim 1, further comprising a motion plate motor coupled to the motion plate, wherein the motion plate motor comprises a motion pin disposed in a motion slot defined in the motion plate.

7. The clamp assembly of claim 1, further comprising a rotatable handle coupled to the plurality of bases.

8. A clamp mechanism, comprising:
a guide, comprising:
a mount hole for a base pin,
a first guide channel, and
a second guide channel;
two opposed bodies comprising hooks, wherein the two opposed bodies are connected with a guide pin such that the two opposed bodies are configured to rotate about the guide pin, wherein the guide pin is disposed through the first guide channel to constrain motion of the guide pin, and wherein the two opposed bodies comprise angled tracks at respective distal ends of each of the opposed bodies;
a return spring configured to apply a return force to the guide pin acting from the guide;
a clamp pin disposed through the angled tracks at the distal ends of each of the opposed bodies and through the secondary guide channel of the guide to constrain motion of the clamp pin.

9. The clamp mechanism of claim 8, further comprising:
a linkage, wherein the clamp pin is mounted to the linkage, and wherein the linkage is configured to move the clamp pin;
wherein movement of the clamp pin from a distal position to a proximal position, pushes against the angled tracks of each opposed body and rotates each opposed body to change a distance between the hooks, wherein changing the distance between the hooks acts to engage or disengage a surgical cassette.

10. The clamp mechanism of claim 9, further comprising a spacer placed over the clamp pin and through the respective angled tracks of each opposed body and between the guide and the linkage.

11. The clamp mechanism of claim 9, further comprising:
a linkage arm assembly comprising a first lever, a second lever, and a lever pin;
wherein the linkage is mounted at an opposing end to the linkage arm assembly with a linkage pin disposed therein to allow relative rotation.

12. The clamp mechanism of claim 11,
wherein the linkage arm assembly is mounted to the base pin;
wherein the linkage arm assembly can extend or compress between the base pin and the linkage pin, and wherein the linkage arm assembly is configured to provide an expansion force from a clamp spring;
herein upward movement of the lever pin acts to rotate the linkages and displace the clamp pin distally to expand the hooks of each opposing body until the clamp pin reaches an end of the angled tracks, wherein further movement of the lever pin moves the hooks distally with a surgical cassette until the surgical cassette contacts a faceplate supporting the base pin, wherein further movement of lever pin compresses the linkage arm assembly between the base pin and the linkage pin and changes a length of the clamp spring.

13. The clamp mechanism of claim 11, further comprising a plate pin on the linkage or the linkage arm assembly, wherein the plate pin is configured to actuate the clamp mechanism.

14. A clamp assembly comprising:
one or more clamp mechanisms of claim 13;
a motion plate in connection to each plate pin of each clamp mechanism;
wherein the motion plate has a motion slot;
a motor;
a drive wheel assembly comprising a motion pin;
wherein the motion pin is engaged in the motion slot
wherein the motor is configured to rotate the motion pin by rotating the drive wheel assembly;
wherein rotation of the motion pin moves the motion plate substantially linearly;
wherein the motion plate actuates the one or more clamp mechanisms.

15. The clamp assembly of claim 14,
wherein the motion slot comprises a grooved bushing that is configured to engage the motion pin of the drive wheel assembly.
